SEQUENCE LISTING (11 sequences)

SEQ ID NO: 1 - DNA, 1613 bp, Mouse
  CDS (64)..(858): Mouse Snail1 encoding sequence
cggagttgactaccgaccttgcgcgacccggtgaccccgactacctaggtcgctctggccaacatgccgcgctccttcctggtcaggaagccgtccgacccccgccggaagcccaactatagcgagctgcaggacgcgtgtgtggagttcaccttccagcagccctacgaccaggcccacctgctggccgccatccctccgcccgaggtcctcaaccccgccgcttcgctgcccaccctcatctgggactctctcctggtaccccaagtgcggccggttgcctgggccaccctcccgctgcgggagagccccaaggccgtagagctgacctcgctgtccgatgaggacagtggcaaaagctcccagccgcccagcccgccctcgccggcgccgtcgtccttctcgtccacctcggcctcgtccctggaggccgaggccttcatcgccttccctggcttgggccaacttcccaagcagctggccaggctctcggtggccaaggacccccagtcgcggaagatcttcaactgcaaatattgtaacaaggagtacctcagcctgggcgctctgaagatgcacatccgaagccacacgctgccttgtgtctgcacgacctgtggaaaggccttctctaggccctggctgcttcagggccacgtccgcacccacactggtgagaagccattctcctgctcccactgcaaccgtgcttttgctgaccgctccaacctgcgtgcccacctccaaacccactcggatgtgaagagataccagtgccaggcctgtgcccgaaccttctcccgcatgtccttgctccacaagcaccaagagtctggctgctccggaggccctcgctgaccctgctacctccccatcctcgctggcatcttcccggagctcaccctcctcctcactgccaggactccttccagccttggtccggggacctgtggcgtccatgtctggacctggttcctgcttggctctcttggtggcctttgccgcaggtggctgatggagtgcctttgtacccgcccagagcctcctacccctcagtattcatgaggtgtagcctctggacacagctgcttcgagccatagaactaaagccaacccactggctgggaagcttgaaccccgctcaggggaccccacttccctacctccctcaaggacccttcaggccaccttctttgaggtacaacagactatgcaatagttcccctcccccccaccccgtccagctgtaaccatgcctcagcagggtggttactggacacatgtccaggtgcccctgggcctgggcaactgtttcagcccccgcccccatttgtcctggtgacacctgtttcacagcagtttaactgtctcagaagggaccatgaataatggccatcacttgttaggggccaagtggggtgcttcagcctggccaatgtgtctcccagaactattttggggcccaacaggtggccccgggagaaagatgtttacattttaaaggtatttatattgtaagcagcattttgtatagttaatatgtacagtttattgatattcaataaaatggttaatttatatactaaaaaaaaaaaaaaaaaaaaaaaaaaaaa

SEQ ID NO: 2 - protein, 264 aa, Mouse
MPRSFLVRKPSDPRRKPNYSELQDACVEFTFQQPYDQAHLLAAIPPPEVLNPAASLPTLIWDSLLVPQVRPVAWATLPLRESPKAVELTSLSDEDSGKSSQPPSPPSPAPSSFSSTSASSLEAEAFIAFPGLGQLPKQLARLSVAKDPQSRKIFNCKYCNKEYLSLGALKMHIRSHTLPCVCTTCGKAFSRPWLLQGHVRTHTGEKPFSCSHCNRAFADRSNLRAHLQTHSDVKRYQCQACARTFSRMSLLHKHQESGCSGGPR

SEQ ID NO: 3 - DNA, 2084 bp, Mouse
  CDS (116)..(925): Mouse Snail2 encoding sequence
cagggagccgggtgacttcagaggcgcctgcctgtcccccgccgcacctgagccaccgcgatgctataggaccgccgcctggaccgttatccgcccgcgcccgcccgcagccaccatgccgcgctccttcctggtcaagaaacatttcaacgcctccaagaagcccaactacagcgaactggacacacacacagttattatttccccatatctctatgaaagttaccctatacctgtcataccaaaaccagagatcctcacctcgggagcatacagccctattactgtatggacatcgtcggcagctccactccactctcctttacccagtggcctttctcctcttactggatactcctcatccttggggcgtgtaagtcccccgccttcctctgacacttcatccaaggatcacagtggttcagaaagtcccattagtgacgaagaggagagactgcagcccaagctttcagacccccatgccatcgaagctgagaagtttcagtgcaatttatgcaataagacctattctacgttctctgggctggccaaacacaagcagctgcactgtgatgcccagtctaggaaatcgttcagctgcaagtactgtgacaaggaatatgtgagcctgggtgccctgaagatgcacattcgaacccacacattgccttgtgtctgcaagatctgtggcaaggctttctccagaccctggctgcttcaaggacacattagaactcacactggggaaaagcctttctcttgccctcactgcaatagggcttttgcagacagatcaaacctgagggcacatctgcagacccactctgatgtaaagaaataccagtgcaaaaactgctccaaaaccttctccagaatgtcgcttctgcataaacatgaggagtctggctgctgtgtggcacactgagtggcgcaaccagtgtttactcaaacagaatgcatttcttcactccaatgacaaatgacaaatgaaagtccaaagacattttctcatgtgcttaccaaccaaatagtatgtataaaaccacaaaagagtcacacacacacacacacacacacacacacacacacacacagagagagagagagagagagagagagagacagacagacagacagacagatacacacacactacagaacagaatctatgtacttaaagttaattcgttctatgtgaagtttaaaattatatttactgacagctagattgaaaggataaaagataagaatctttctctttaaagatgaagtgaaaagcacattgcatcttttcttactaagaaagaatacagagatttacactgctgccaaaccatttcaaccaaaggaacagtatttcttcttaatagaattgtaatagtgtttccaagaggaagagagtctgccagacactatctcaggtgccttataaagtactccaagtttacttccttaaatgtatgatgcctggttgtcatcagtgaatgacagccttttctggattacctacaatgttttaaaactatattgttaagagaaaaaaaaccaaaaacaagaaaaagaacagaacacaagagaatgtattaaagtattcttgttttatttttgccatgtgtgccttggaagaggagggaaagacaaacttcaaacattcctggtgcgtgtcccatgtctttctttttaaaaaagaatcttaatgttttataatacaaagtaatgaaaatgtgcaaaagaatttcttagacattcagtaatgtacttagacttttgaaaattcatgtgatggatgcagtaatacaatgcccctccaagtgcctgtcttaatgacttgtgtagttgatgaactgatgtaaatttgtgtttatttttatacaactgaatgaactctgtatgaaagtgaggtacggttaatagccacgcctatattcaaccagaatacttgtgaaatcaatgtccttttttaaaaagtaactttcaaggtctcttttttacaataaacatttttgagtaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 269 aa, Mouse
MPRSFLVKKHFNASKKPNYSELDTHTVIISPYLYESYPIPVIPKPEILTSGAYSPITVWTSSAAPLHSPLPSGLSPLTGYSSSLGRVSPPPSSDTSSKDHSGSESPISDEEERLQPKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 5 - DNA, 1708 bp, Homo sapiens
  CDS (71)..(865): Human Snail1 encoding sequence
ggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

SEQ ID NO: 6 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 7 - DNA, 2101 bp, Homo sapiens
  CDS (165)..(971): Human Snail2 encoding sequence
agttcgtaaaggagccgggtgacttcagaggcgccggcccgtccgtctgccgcacctgagcacggcccctgcccgagcctggcccgccgcgatgctgtagggaccgccgtgtcctcccgccggaccgttatccgcgccgggcgcccgccagacccgctggcaagatgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcacattgcatcttttcttcctaaaaaaatgcaaagatttacattgctgccaaatcatttcaactgaaaagaacagtattgctttgtaatagagtctgtaataggatttcccataggaagagatctgccagacgcgaactcaggtgccttaaaaagtattccaagtttactccattacatgtcggttgtctggttgccattgttgaactaaagcctttttttgattacctgtagtgctttaaagtatatttttaaaagggaggaaaaaaataacaagaacaaaacacaggagaatgtattaaaagtatttttgttttgttttgtttttgccaattaacagtatgtgccttgggggaggagggaaagattagctttgaacattcctggcgcatgctccattgtcttactattttaaaacattttaataatttttgaaaattaattaaagatgggaataagtgcaaaagaggattcttacaaattcattaatgtacttaaactatttcaaatgcataccacaaatgcaataatacaataccccttccaagtgcctttttaaattgtatagttgatgagtcaatgtaaatttgtgtttatttttatatgattgaatgagttctgtatgaaactgagatgttgtctatagctatgtctataaacaacctgaagacttgtgaaatcaatgtttcttttttaaaaaacaattttcaagttttttttacaataaacagttttgatttaaaatctcgtttgtatactattttcagagactttacttgcttcatgattagtaccaaaccactgtacaaagaattgtttgttaacaagaaaaaaa

SEQ ID NO: 8 - protein, 268 aa, Homo sapiens
MPRSFLVKKHFNASKKPNYSELDTHTVIISPYLYESYSMPVIPQPEILSSGAYSPITVWTTAAPFHAQLPNGLSPLSGYSSSLGRVSPPPPSDTSSKDHSGSESPISDEEERLQSKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 9 - DNA, 19 bp, Homo sapiens
gatgcacatccgaagccac

SEQ ID NO: 10 - DNA, 27 bp, Artificial sequence
  Oligo A
ggtaccatctacacattcactactaga

SEQ ID NO: 11 - DNA, 27 bp, Artificial sequence
  Oligo B
acgcgttttccaaggacggaaaaagaa

(19) United States
(12) Patent Application Publication
(10) Pub. No.: US 2010/0286231 A1
(43) Pub. Date: Nov. 11, 2010

(54) METHOD FOR IDENTIFYING A RENAL FIBROSIS PROCESS USE OF SNAIL- ACTIVITY-INHIBITING COMPOUNDS IN THE PRODUCTION OF PHARMACEUTICAL COMPOSITIONS, METHOD FOR IDENTIFYING SAID INHIBITING COMPOUNDS, SAID PHARMACEUTICAL COMPOSITIONS AND APPLICATIONS THEREOF

(75) Inventors: Maria Angela Nieto Toledano, San Juan (Alicante) (ES); Agnes Boutet, San Juan (Alicante) (ES); Cristina Alvarez De Frutos, San Juan (Alicante) (ES)

Correspondence Address: DARBY & DARBY P.C., P.O. BOX 770, Church Street Station, New York, NY, 10008-0770, US

(73) Assignee: Universidad Miguel Hemandez de Elche, Elche (ES)

(21) Appl. No.: 12/161,450

(22) PCT Filed: Oct. 30, 2006
(86) PCT No.: PCT/ES06/70165

§ 371 Date: Feb. 2, 2009

(30) Foreign Application Priority Data
Jan. 19, 2006 (ES) P200600119

(51) Int. Cl. A61K 31/7088; C12Q 1/68; C12Q 1/02; G01N 33/53; C07H 21/02; A61P 13/12

(52) U.S. Cl. 514/44 A; 435/6; 435/29; 435/7.1; 514/44 R; 536/23.1; 536/24.5

(57) ABSTRACT

The present invention relates to Snail, which is a protein that is directly involved in the etiopathogeny of renal fibrosis, in addition to being a marker of this disease. Therefore, the identification thereof may be used as a diagnosis for renal fibrosis. On the other hand, the Snail protein may be of great utility in identifying new drugs for the treatment of renal fibrosis, and its gene inhibition may be of use as a form of treatment in accordance with the present invention.

This application is a U.S. national phase application under 35 U.S.C. §371 of International Patent Application No. PCT/ES2006/070165 filed Oct. 30, 2006, which claims the benefit of priority to the Spanish Application No. P200600119 filed Jan. 19, 2006, the disclosures of all of which are hereby incorporated by reference in their entireties. The International Application was published in Spanish on Jul. 26, 2007 as WO 2007/082967.

### FIELD OF THE INVENTION

This invention belongs to the field of biomedicine and, more specifically, to the application of biotechnological tools for the diagnosis and treatment of human diseases and, more specifically, of renal fibrosis.

### BACKGROUND

The renal epithelium originates from cells that undergo a mesenchyme-epithelium transition (MET). The reverse process, epithelium-mesenchyme transition (EMT) has been implicated in the progression of epithelial tumours and in the fibrosis which ultimately leads to renal failure. Snail transcription factors induce both physiological and pathological EMTs by the repression of E-Cadherin transcription (amongst other targets) (Cano et al., 2000; Bathe et al., 2000; Bolos et al., 2003). It has been determined that Snail also suppresses the expression of kidney-specific cadherin, Cadherin 16 (Thompson et al., 1995), by repressing the transcription of the activator thereof, HNF-1 beta (Hepatic nuclear factor-1 beta; Bai et al., 2002). This repression is active during early embryo development and it has been observed that the disappearance of Snail is concomitant with the appearance of HNF-1 beta and, subsequently, of Cadherin 16, which are identified as signs of differentiation of the renal epithelium (Dressler, 2002). Snail activation in the mature kidney may be considered as a return to embryogenic properties and has been identified as sufficient to induce EMT and renal fibrosis in mice.

Progressive renal fibrosis is a devastating disease that triggers renal failure in patients suffering from various diseases, such as glomerulonephritis, IgA nephropathy, diabetes, renal damage induced by toxicity, urinary obstruction or deterioration of kidney transplants, amongst others (Liu, 2004; Kalluri and Neilson, 2003, Zeisberg and Kalluri, 2004; Vongwiwatana et al., 2005). It used to be believed that renal fibrosis arose from the activation of interstitial fibroblasts, but there are conclusive data which suggest that it also arises as a result of an EMT taking place in renal tubules' epithelial cells (Iwano et al., 2002). Signs of tubular EMT have been observed in the kidneys of patients with renal fibrosis (Jinde et al., 2001; Rastaldi et al., 2002) and, in animal models it has been calculated that about 36% of the fibroblast population arises from a local EMT of epithelial cells (Kalluri and Neilson, 2003).

On the other hand, Snail expression is concomitant with EMT under-gone in the kidney following unilateral ureteral obstruction model that causes renal fibrosis (Sato et al., 2003); although, a cause-effect has not been established, since the cause of renal fibrosis was the ureteral obstruction. Furthermore, Snail expression is concomitant with the EMT of mesothelial cell involved in mesothelial fibrosis in the dialisates of patients subject to peritoneal dialysis and in culture cells treated with TGF-beta (Yáñez-Mo et al. 2003).

The use of BMP-7 as an inhibitory agent with the capacity to reverse renal fibrosis is patented. BMP-7 (bone morphogenic protein number 7) exerts this effect due to its capacity to inhibit TGF-beta, an agent that induces renal fibrosis and is known to induce Snail (reviewed in Barrallo-Gimeno and Nieto, 2005; United States Patent Application 20020173453, Method of treating renal injury). Both TGF-beta and BMP-7 are extracellular signalling molecules which initiate a complex cascade of events. If the induction of Snail is sufficient to reproduce renal fibrosis, the specific inhibition of Snail is expected to be a much more precise therapy than the inhibition of the entire TGF-beta signalling cascade.

Finally, if Snail activity in the mature kidney is sufficient to induce EMT and renal fibrosis, the presence of Snail could be considered to be a marker of renal fibrosis; the inhibition thereof, could be considered as a form of anti-fibrotic therapy, and Snail could be of great utility in identifying new anti-fibrotic drugs.

### SUMMARY OF THE INVENTION

An object of this invention is a method for identifying a renal fibrosis in humans, hereinafter renal fibrosis identification method of the invention, based on the identification of the presence of Snail in a biological sample, which comprises the following steps:

a) identification of the presence of Snail, in a biological sample of renal origin, and

b) comparison of the presence of Snail observed in Step a) with the absence thereof in a control sample, and where its presence is indicative of the existence of renal fibrosis.

A particular object of the invention is the identification method of the invention, wherein the identification of Snail of step a) relates to the human forms of Snail1 (hSnail1, SEQ ID NO 5 and 6) or Snail 2 (hSnail2, SEQ ID NO 7 and 8), whether the identification is in the form of a gene transcript (mRNA) or the protein form of both genes.

Another object of this invention is a method of identifying and evaluating the activity of Snail protein inhibitory compounds which are useful in the treatment of renal fibrosis, hereinafter compound identification method of this invention, which comprises the following steps:
a) Incubating, under suitable conditions, a candidate compound of this invention with a biological system exhibiting Snail expression that produces renal fibrosis,
b) measuring an indicative parameter of renal fibrotic, and
c) Identifying a compound that inhibits Snail protein activity when a reduction of said renal fibrosis parameter is observed.

Another particular object of this invention is the identification method of the invention where the biological system of Step a) is a transgenic animal where the expression of the Snail protein is inducible in a constant or conditional manner, and where the expression thereof causes renal fibrosis. A particular embodiment is one wherein the transgenic animal is the transgenic mouse of this invention (Example 2, transgSnail1-ER mouse).

Another object of this invention is the use of a compound or agent that inhibits Snail protein activity, hereinafter use of a compound of this invention, in the preparation of drugs or pharmaceutical compositions for the treatment of renal fibrosis, preferably in humans.

Therefore, in another particular embodiment of the invention, the use of a compound is based on the fact that the inhibitory compound is a nucleic acid or polynucleotide which prevents or reduces the expression of the human Snail protein encoding gene and includes a nucleotide sequence selected from:

a) a Snail protein gene or mRNA sequence specific anti-sense nucleotide sequence,

b) a Snail protein mRNA specific ribozyme,

c) a Snail protein mRNA specific aptamer, and

d) a Snail protein mRNA specific interference RNA (iRNA).

A particular embodiment of the invention is the use of an iRNA which preferably binds to the Snail mRNA gatgcacatccgaagccac (SEQ ID NO 9) fragment sequence or to another fragment that comprises the latter.

Another object of this invention is a pharmaceutical composition or a drug for the treatment of renal fibrosis, hereinafter pharmaceutical composition of this invention, which comprises a therapeutically effective quantity of a compound or agent that inhibits the Snail protein, jointly and optionally with, one or more pharmaceutically acceptable adjuvants and/or vehicles.

A particular embodiment of the invention is a pharmaceutical composition wherein the inhibitory compound is a nucleic acid or polynucleotide which prevents or reduces the expression of the human Snail protein encoding gene and includes a nucleotide sequence selected from:

a) a Snail protein gene or mRNA sequence specific anti-sense nucleotide sequence,

b) a Snail protein mRNA specific ribozyme,

c) a Snail protein mRNA specific aptamer, and

d) a Snail protein mRNA specific interference RNA (iRNA).

Another object of this invention is the use of the pharmaceutical composition of the invention in a treatment method for a mammal, preferably a human being, suffering from renal fibrosis, hereinafter use of the pharmaceutical composition of this invention, which consists of administering the therapeutic composition that inhibits the fibrotic process.

A particular object of this invention is the use of the pharmaceutical composition of this invention wherein the renal fibrosis is caused by a disease, disorder or pathology which, for illustrative purposes and without this limiting the scope of the invention, belongs to the following group: glomerulonephritis, IgA neuropathy, diabetes, renal damage induced by toxicity, urinary obstruction and deterioration of kidney transplants.

### BRIEF DESCRIPTION OF THE DRAWINGS

FIG. 1. Snail induces EMT in NMuMG cells concomitant with Cadherin-16 repression. Phase-contrast images (a, b), expression of E-Cadherin (c, d) and F-actin (e, f) in cells transfected with the empty vector (Mock) or transfected with Snail1 (Snail). The motility was determined by a cultured wound assay (g, h). The invasive properties were analysed by the cells' capacity to go through collagen IV gels. (k) Expression of E-Cadherin, cadherin-16 and Snail1 by RT-PCR in Mock and Snail cells. The cells that express Snail have lost E-Cadherin, reorganised the actin fibres, and acquired a fibroblastic morphology, all of which is indicative of an EMT. Moreover, they are capable of closing the wound in 24 hours and of invading the collagen gels. Bar scale 50 μm.

FIG. 2. The renal epithelia that express Cadherin-16 originate from positive mesenchyme for Snail genes. In situ hybridization for Cadherin-16, Snail1 and Snail2 on different days of the mouse's embryo development: 10.5 (a-i), 13.5 (j-o) and 17.5 (p-u). Cadherin-16 is expressed in the newly-formed nephric duct epithelium (b, nd), which no longer expresses Snail (e, h, inserts). Snail is observed in the undifferentiated metanephric mesenchyme (mm). The desiccated urogenital systems (j insert) or the sections thereof (j-o) were hybridised with probes in order to detect Cadherin-16 and the Snail genes. Cadherin-16 is also expressed in the mounds in formation (j, k), jointly with the sexual ducts (sd) and the transient mesonephros' epithelial mounds (ms), as shown in the insert in j. The Snail genes continue to be expressed in the remaining mesenchyme (mm, l-o). The nephrons' and the collecting tubules' epithelium expresses high Cadherin-16 levels when the entire mesenchyme has become differentiated into the epithelium and Snail gene expression has disappeared (r-u). This expression remains in the adult kidney. Bar scale 100 μm. v. Snail transcription factors inhibit Cadherin-16 gene promoter activity. Snail1 and Snail2 continue to repress promoter activity when the Snail binding sites have been eliminated.

FIG. 3. The expression of HNF-1beta precedes that of Cadherin-16 in the developing kidney's epithelial components. In situ hybridization of embryos with 10.5, 13.5 and 17.5 days of development and their corresponding sections. HNF-1 beta expression is observed as soon as mesenchyme-derived epithelia appear. At 10.5 days of development, the expression is observed in the nephric duct (b) and in the mesenchyme that is condensing in regions where Cadherin-16 expression has vet not appeared (FIG. 2c-cm). At 13.5 days of development, in addition to the nephric duct, many HNF-1beta, expression sites are observed (d), some of which already express Cadherin-16. Snail2 is expressed in the metanephric mesenchyme. Bar scale 100 μm

FIG. 4. Snail1 and Snail2 repress Cadherin-16 expression by repressing the transcription of the activator thereof, HNF-1beta. a) The activation of Snail1 represses the expression of Cadherin-16 and HNF-1beta. The NMuMG cells stably transfected with an activatable version of Snail1 change their morphology 24 hours after the induction. b) Transgene expression by RT-PCR. c) Real-time RT-PCR of Cadherin-16 and HNF-1beta 24 hours after administration of the inducer, 4-OH-tamoxifen. And d) Diagram of the 1-kb region in front of the mouse HNF-1 beta gene translation initiation site, showing a very conserved region with humans. Both Snail binding sites are indicated by black boxes. Snail1 and Snail2 repress the HNF-1 beta native promoter activity, but did not affect the activity of a promoter whose conserved site was eliminated.

FIG. 5. Snail1 represses in vivo expression of HNF-1beta and cadherin-16. a, b) The exogenous Snail protein was translocated to the nucleus following the administration of tamoxifen, as observed by the anti-human-estrogen receptor antibody. c) Real-time RT-PCR of Snail1 in normal and transgenic mice in the absence or presence of tamoxifen. Expression of HNF-1beta (d, e), cadherin-16 (g, h) and Snail2 (j, k) in marrow sections of two-week-old transgenic kidneys and the corresponding expression values by real-time RT-PCR (f, i and l, respectively). Bar scale 25 μm. Although it is not shown, E-Cadherin, a direct target of Snail shown in tissues with a different etiology, also disappears (reviewed in Barrallo-Gimeno and Nieto, 2005).

FIG. 6. Snail activation is sufficient to induce renal fibrosis in transgenic mice. Snail activation induces a morphological change characteristic of an EMT (a, b); activation of the mesenchymal marker vimentin (c, d); activation of Collagen I gene transcription (e, f) and deposition of collagen fibres, as observed by means of the Trichrome-Masson stain (g, h).

FIG. 7. Snail activation also produces fibrosis in the renal cortex. The same morphological changes indicative of EMT are observed as in the marrow; also observed is the disappearance of Cadherin-16 and E-Cadherin, and the appearance of Snail2 and of Collagen 1 deposits.

FIG. 8. Human kidneys with fibrosis show strong Snail expression. Tissues from normal human kidneys and from patients with pulmonary fibrosis subject to nephrectomy for renal tumour exeresis and for urinary obstruction and renal failure, respectively, were analysed. Shown is an RT-PCR quantitative analysis of Snail1 and Snail2 expression in normal human kidney tissue (C=control, n=4), non-fibrotic tissue from a kidney with fibrosis (1C) and fibrotic tissue from patient 1 (1F) and patient 2 (2F). The transcription levels were normalised with the GAPDH mRNA expression levels and the error bars represent the standard error from the mean.

### DETAILED DESCRIPTION OF THE INVENTION

This invention is based on the fact that the inventors have observed that Snail genes repress in vivo expression of cadherin-16 by indirectly repressing the gene transcription of the activator thereof, HNF1beta, in both cellular and animal models. In order to determine whether both Snail1 and Snail2 genes may repress cadherin-16 in vivo, their relative expression patterns were studied during embryo development in mice, when different EMT and MET processes take place that lead to the formation of the mature kidney (Example 1), in transgenic mice with inducible expression of the Snail genes (Example 2), and in samples from patients with renal fibrosis.

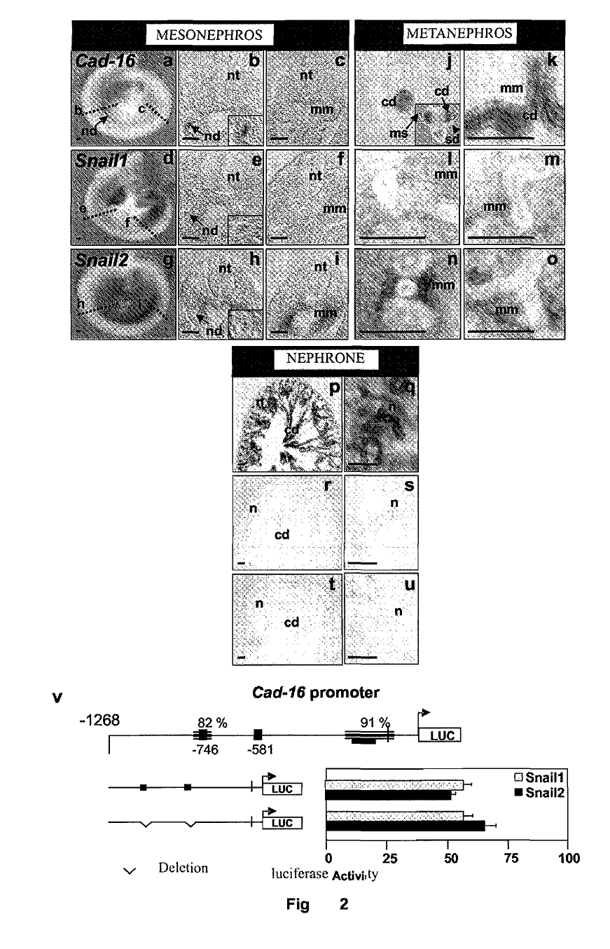

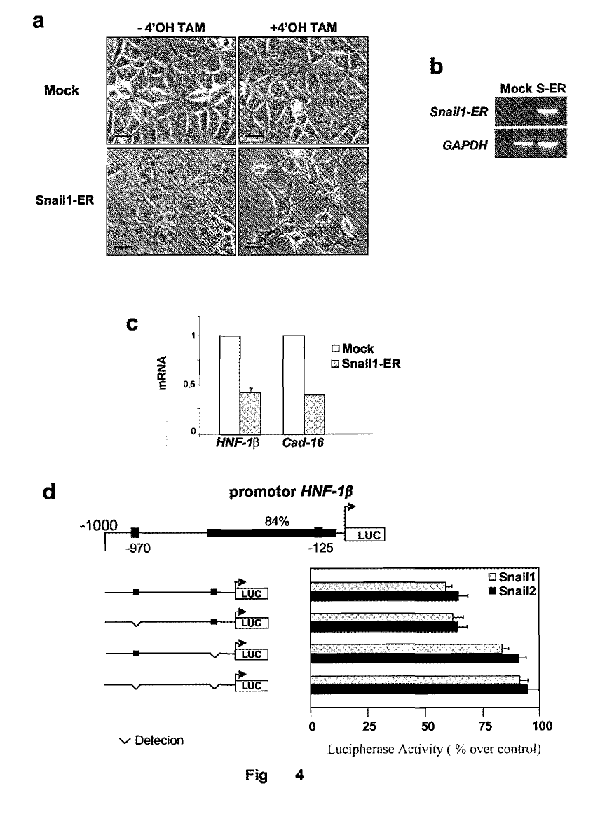

The data show that the expression patterns are complementary in the different embryo stages and that cadherin-16 only appears in the mesenchyme following the disappearance of expression of the Snail1 and Snail2 genes (Example 1). These data are consistent with the fact that Snail indirectly represses in vivo expression of the cadherin-16 gene (FIG. 2v), through the repression of HNF-1beta and, more specifically, through direct action on the promoter thereof (binding to conserved consensus E-box identified in this invention), thereby inducing a complete EMT (FIGS. 4a and 4c).

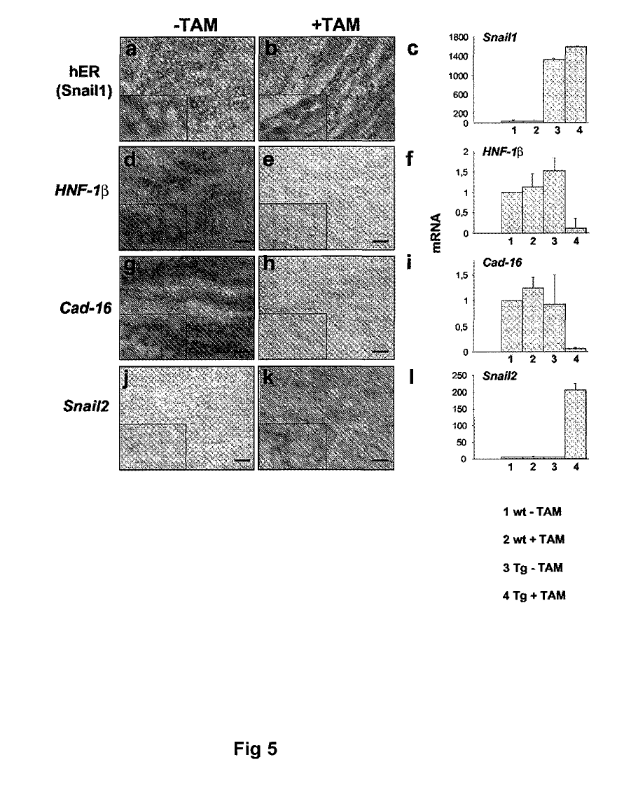

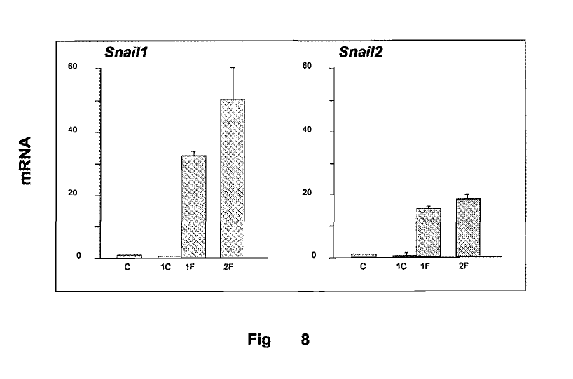

Moreover, in order to determine whether the Snail genes may repress HFN-1 beta transcription and, consequently, Cadherin-16 expression, transgenic mice with inducible Snail1 activity were generated (Example 2). Thus, it was observed that Snail1 represses HNF-1 beta in vivo, which induces repression of cadherin-16, and induces the loss of the cells' epithelial characteristics, which seem to acquire a morphology similar to the fibroblastic morphology that occurs in a complete EMT (FIGS. 5b, e, h, k, inserts). These changes disclosed herein are reminiscent of those observed following the experimental induction of renal fibrosis under different conditions (Liu, 2003). Finally, the results in patients with renal fibrosis showed that Snail expression causes the epithelium-to-mesenchyme transition (Example 3, FIG. 8).

In summary, these data suggest that the Snail1 and Snail2 genes act as repressors of the epithelial phenotype in the mature kidney and, moreover, that the activation thereof is sufficient to induce all the characteristics of EMT and of renal fibrosis, i.e., that there is a direct relationship—not only a temporal association—between the activity of the Snail genes and the etiopathogeny of this disease. Thus, the presence of Snail may be considered to be a marker of renal fibrosis and, therefore, the identification thereof may be used as a diagnosis of renal fibrosis; and, on the other hand, the Snail protein may be of great utility in identifying new drugs for the treatment of renal fibrosis and the gene inhibition thereof as a form of therapy. These therapeutic approaches to renal fibrosis are based on the use of compounds or agents that inhibit the activity of the Snail protein.

Therefore, an object of this invention is a method for identifying a renal fibrosis hereinafter renal fibrosis process identification method of the invention, based on the identification of the presence of Snail in a biological sample, which comprises the following steps:

a) identification of the presence of Snail, in a biological sample of renal origin, and

b) comparison of the presence of Snail observed in Step a) with the absence thereof in a control sample, and where its presence is indicative of the existence of renal fibrosis.

As used in this invention, the term “Snail genes” or “Snail proteins” refers to both the Snail1 gene or protein (SEQ ID NO 1 and 2, respectively) and the Snail2 gene or protein (SEQ ID NO 3 and 4, respectively), as well as any nucleotide or amino acid (aa) sequence that is analogous to those of other species, respectively. In the sense used in this description, the term “analogous” is intended to include any nucleotide or amino acid sequence that may be isolated or constructed on the basis of the nucleotide or aa sequences shown in this specification, for example, by the introduction of conservative or non-conservative nucleotide or aa substitutions, including the insertion of one or more nucleotides or aa, the addition of one or more nucleotides or aa at any of the ends of the molecule or the deletion of one or more nucleotides or aa, at any end or in the interior of the sequence, and which is an encoding sequence or peptide with an activity similar to that of the sequences of the invention, i.e., that it is capable of inducing renal fibrosis.

In general, an analogous nucleotide or amino acid sequence is substantially homologous to the amino acid sequence previously discussed. In the sense used in this description, the expression “substantially homologous” means that the nucleotide or aa, sequences in question have a degree of homology of, at least, 40%, preferably of, at least, 85%, or more preferably of, at least, 95%.

A particular object of the invention is the identification method of the invention wherein the identification of Snail of Step a) relates to the human forms of Snail1 (hSnail1, SEQ ID NO 5 and 6) and Snail2 (hSnail2, SEQ ID NO 7 and 8), whether the identification is in the form of a gene transcript (mRNA) or the protein form of both genes. These analyses designed to identify Snail expression levels may be performed by a person skilled in the field of biomedicine, thanks to the information disclosed in this invention and in the state of the art by different techniques (Sambrook, J., Fritsch, E. F., and Maniatis, T. (1989). Molecular cloning: a laboratory manual, 2nd ed. Cold Spring Harbor Laboratory, Cold Spring Harbor, N.Y.).

Another particular object of the invention is the renal fibrosis identification method wherein the identification of Snail is performed using specific Snail antibodies. The antibodies may be monoclonal or polyclonal.

Another particular object of the invention is the renal fibrosis identification method wherein the identification of Snail is performed by in situ hybridization with a Snail precursor.

Another particular object of the invention is the renal fibrosis identification method wherein the identification of Snail is performed by RT-PCR of a Snail gene precursor. This method is based on the extraction of polyA+RNA from a biological sample of renal origin and a control tissue with the amplification of the Snail-encoding sequence with suitable primer oligonucleotides.

On the other hand, this diagnostic method for renal fibrosis may be performed using Snail as the sole marker or jointly with other markers of renal fibrosis, for example as a part of a biological expression microarray, either in gene form—from mRNA—or in protein form, that defines a diagnostic marker or profile for pulmonary fibrosis.

Another object of this invention is a method of identifying and evaluating the activity of Snail protein inhibitory compounds which are useful to treat renal fibrosis, hereinafter compound identification method of this invention, which comprises the following steps:
a) incubating, under suitable conditions a candidate compound of the invention with a biological system exhibiting Snail expression that produces renal fibrosis,
b) measuring an indicative parameter of the renal fibrosis process, and
c) identifying a compound that inhibits Snail protein activity when a reduction of the renal fibrosis parameter is observed.

Another particular object of this invention is the identification method of the invention where the biological system of Step a) is a transgenic animal where the expression of the Snail protein is inducible in a constant or conditional manner and where the expression thereof causes renal fibrosis. A particular embodiment is one wherein the transgenic animal is the transgenic mouse of this invention (Example 2, transgSnail1-ER mouse).

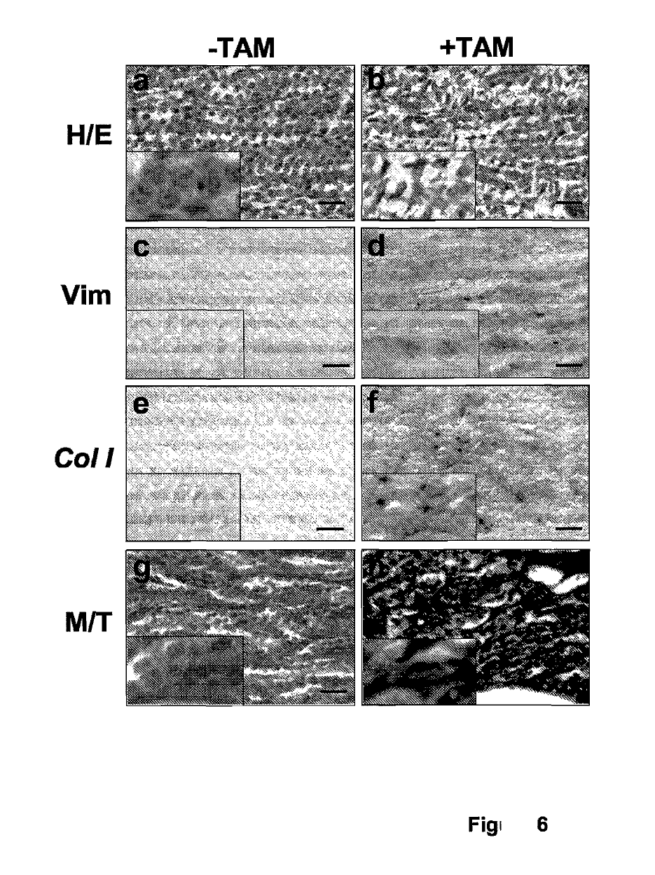

Another particular object of this invention is the identification method of the invention wherein the parameter related to the renal fibrosis process of Step a) belongs, for illustrative purposes and without this limiting the scope of this invention, to the following group: a morphological change characteristic of an EMT, level of vimentin, Collagen I gene transcription, and deposition of collagen fibres (FIG. 6, Example 2).

Another object of this invention is the use of a compound or agent that inhibits Snail protein activity, hereinafter use of a compound of this invention, in the preparation of drugs or pharmaceutical compositions for the treatment of renal fibrosis, preferably in humans.

As used in this invention, the term “inhibitory or antagonist compound/agent” refers to a molecule which, when it is bound to or interacts with the Snail protein (for example, SEQ ID NO 2, SEQ ID NO 4, SEQ ID NO 6 and SEQ ID NO 8), or with functional fragments thereof, reduces or eliminates the intensity or the duration of the biological activity of the protein. This definition includes, furthermore, those compounds which prevent or reduce the expression of the Snail protein encoding gene, i.e., which prevent or reduce gene transcription, mRNA maturation, mRNA translation and post-translational modification. An inhibitory agent may be composed of a peptide, a protein, a nucleic acid or polynucleotide, a carbohydrate, an antibody, a chemical compound, or any other type of molecule that reduces or eliminates the effect and/or the function of the Snail protein.

For illustrative purposes, said polynucleotide may be a polynucleotide that encodes a Snail protein gene or mRNA sequence specific anti-sense nucleotide sequence, or a polynucleotide that encodes a Snail protein mRNA specific ribozyme, or a polynucleotide that encodes a Snail protein mRNA specific aptamer, or a polynucleotide that encodes a Snail protein mRNA specific interference RNA (“small interference RNA” or siRNA).

The above-mentioned polynucleotides may be used in a gene therapy process which allows, by means of any technique or procedure, the integration thereof in the cells of a human patient. This objective may be achieved by administering a gene construct comprising one of the above-mentioned polynucleotides to these kidney cells in order to transform said cells, allowing for their expression in the interior thereof in such a way that Snail protein expression is inhibited. Advantageously, the gene construct may be included within a vector, such as, for example, an expression vector or a transfer vector.

As used in this invention, the term “vector” refers to systems used in the process of transferring an exogenous gene or an exogenous gene construct inside the cell, thereby allowing for the transport of exogenous genes and gene constructs. The vectors may be non-viral or viral vectors (Pfeifer A, Verma IM (2001) Gene therapy: promises and problems. Annu Rev Genomics Hum Genet 2: 177-211), and the administration thereof may be prepared by a person skilled in the art on the basis of the needs and specificities of each case.

Therefore, in another particular embodiment of the invention, the use of a compound is based on the fact that the inhibitory compound is a nucleic acid or polynucleotide which prevents or reduces the expression of the human Snail protein encoding gene and includes a nucleotide sequence selected from:

a) a Snail protein gene or mRNA sequence specific anti-sense nucleotide sequence,

b) a Snail protein mRNA specific ribozyme,

c) a Snail protein mRNA specific aptamer, and

d) a Snail protein mRNA specific interference RNA (iRNA).

Previously, antisense oligonucleotides have been disclosed (US20060003956, Materials and methods for the derepression of the E-cadherin promoter; Kajita M, McClinic K N, Wade P A. Aberrant expression of the transcription factors snail and slug alters the response to genotoxic stress. Mol Cell Biol. 2004, 24(17): 7559-66); also disclosed and patent protected are siRNAs that inhibit the expression thereof (Peinado H, Del Carmen Iglesias-de la Cruz M, Olmeda D, Csiszar K, Fong K S, Vega S, Nieto M A, Cano A, Portillo F. A molecular role for lysyl oxidase-like 2 enzyme in snail regulation and tumor progression. EMBO J. 2005, 24(19): 3446-58; Tripathi M K, Misra S, Chaudhuri G. Negative regulation of the expressions of cytokeratins 8 and 19 by SLUG repressor protein in human breast cells. Biochem Biophys Res Commun. 2005, 329(2): 508-15). On the other hand, these gene inhibition techniques and, more specifically, transport of the compounds—antisense oligonucleotides, iRNA, ribozymes or aptamers—may be performed using nanoparticles, which increase the success rate of the transfer (Lu PV and Woodle M C, Adv Genet 54: 117-42, 2005; Hawker C J and Wooley K L, Science 19 (309): 1200-5, 2005).

Thus, a particular embodiment of the invention is the use of an iRNA that preferably binds to the gatgcacatccgaagccac (SEQ ID NO 9) Snail mRNA fragment sequence or to another fragment which comprises the latter.

Nucleotide sequences over Steps a)-d) mentioned above prevent mRNA gene expression or mRNA expression in the Snail protein, and, therefore, destroy its biological function, and may be developed by a person skilled in the field of genetic engineering on the basis of the existing knowledge about transgenesis and gene expression destruction in the state of the art (Clarke, A. R. (2002) Transgenesis Techniques. Principles and Protocols, 2nd Ed. Humana Press, Cardiff University; U.S. Pat. No. 7,368,436. Gleave, Martin. TRPM-2 antisense therapy; Puerta-Fernández E et al. (2003) Ribozymes: recent advances in the development of RNA tools. FEMS Microbiology Reviews 27: 75-97; Kikuchi, et al., 2003. RNA aptamers targeted to domain II of Hepatitis C virus IRES that bind to its apical loop region. J. Biochem. 133, 263-270; Reynolds A. et al., 2004. Rational siRNA design for RNA interference. Nature Biotechnology 22 (3): 326-330).

On the other hand, the origin of these compounds that inhibit Snail protein activity may be varied, such that they may be of natural origin (for example, vegetable, bacterial, viral, animal origin, or from eukaryotic microorganisms) or synthetic.

Another object of this invention is a pharmaceutical composition or a drug for the treatment of renal fibrosis, hereinafter pharmaceutical composition of this invention, which comprises a therapeutically effective quantity of a compound or agent that inhibits the Snail protein, jointly with, optionally, one or more pharmaceutically acceptable adjuvants and/or vehicles.

A particular embodiment of this invention is a pharmaceutical composition wherein the inhibitory compound is a nucleic acid or polynucleotide which prevents or reduces the expression of the human Snail protein encoding gene and includes a nucleotide sequence selected from:

a) a Snail protein gene or mRNA sequence specific anti-sense nucleotide sequence,

b) a Snail protein mRNA specific ribozyme,

c) a Snail protein mRNA specific aptamer, and

d) a Snail protein mRNA specific interference RNA (iRNA).

As previously discussed, a particular embodiment of the invention is the pharmaceutical composition of the invention wherein the Snail inhibitor is an iRNA that preferably binds to the gatgcacatccgaagccac (SEQ ID NO 5) Snail mRNA fragment sequence or to another fragment that comprises the latter.

The pharmaceutically acceptable adjuvants and vehicles that may be used in the compositions are the adjuvants and vehicles known by those skilled in the art and habitually used in the preparation of therapeutic compositions.

In the sense used in this description, the expression “therapeutically effective quantity” refers to the quantity of the agent or compound that inhibits Snail protein activity calculated to produce the desired effect and, in general, will be determined, amongst other factors, by the compounds' characteristics, including the patient's age, condition, the severity of the alteration or disorder, and the administration route and frequency.

In a particular embodiment, the therapeutic composition is prepared in solid form or in aqueous suspension in a pharmaceutically acceptable diluent. The therapeutic composition provided by this invention may be administered by any suitable administration route; to this end, the composition will be formulated in the pharmaceutical form suitable for the chosen administration route. In a particular embodiment, the administration of the therapeutic composition provided by this invention is performed by parenteral route, by oral route, by intraperitoneal route, by subcutaneous route, etc. A review of the different pharmaceutical forms to administer drugs and the necessary excipients to obtain them may be found, for example, in “Tratado de Farmacia Galénica”, C. Fauli i Trillo, 1993, Luzán 5, S. A. Ediciones, Madrid.

Another object of this invention is the use of the pharmaceutical composition of the invention in a treatment method for a mammal, preferably a human being, suffering from renal fibrosis, hereinafter use of the pharmaceutical composition of this invention, which consists of administering the therapeutic composition that inhibits the fibrosis process.

A particular object of this invention is the use of the pharmaceutical composition of this invention wherein the renal fibrosis is caused by a disease, disorder, or pathology which, for illustrative purposes, and without limiting the scope of the invention, belongs to the following group: IgA nephropathy, glomerulonephritis, diabetes, renal damage induced by toxicity, urinary obstruction and deterioration of kidney transplants.

### EXAMPLES OF THE INVENTION

### Example 1

### Snail Genes Repress Cadherin-16 Expression by Repressing the Gene Transcription of the Activator Thereof, HNF1-Beta

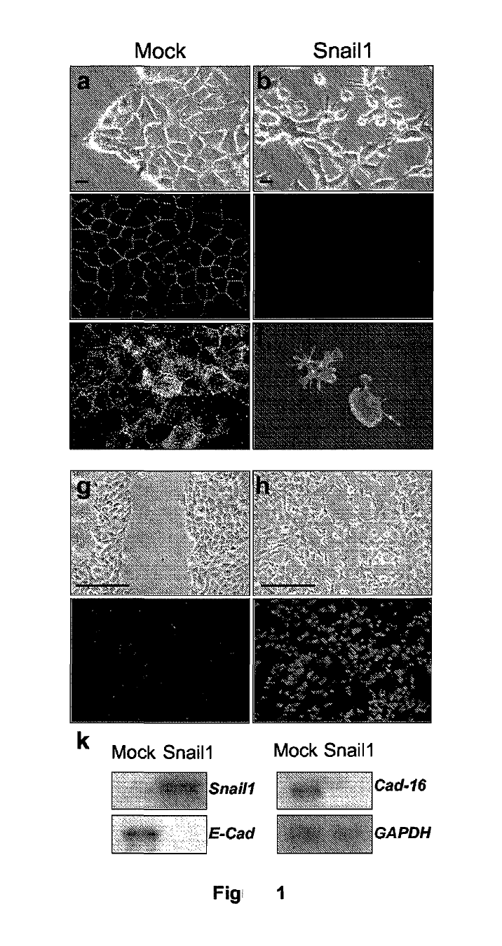

The ectopic expression of Snail1 in the NMuMG cell line induced an EMT, as has been previously described in other epithelial cell lines (Cano et al., 2000; Bathe et al., 2000), with the acquisition of mesenchymal characteristics, such as the loss of E-cadherin expression (FIG. 1d), the reorganisation of the F-actin cytoskeleton (FIG. 10; moreover, they acquired mobility (FIGS. 1g and 1h) and invasive properties (FIGS. 1i and 1j). In addition to repressing known epithelial markers, it also repressed the expression of Cadherin-16 (FIG. 1k), a kidney-specific cadherin. Since the mouse breast and other cell lines from human breast did not express Cadherin-16, it was concluded that this expression is particular to the NMuMG cell line, but it induced us to study whether Snail can repress Cadherin-16 in the kidney.

In order to determine whether both Snail genes can repress Cadherin-16 in vivo, the relative expression patterns were studied during embryo development in mice when different EMT and MET processes take place leading to the formation of the mature kidney. As soon as the nephric ducts epithelialise, Snail2 expression is repressed and cadherin-16 clearly appears; it was observed that the antero-posterior gradient differentiation in the ducts correlates with the complementary expression of the cadherin-16 and Snail genes (FIG. 2a-2i). The collecting ducts induced the nephric mesenchyme to condense and differentiate in tubular structures which will give rise to the nephrons. The nephric mesenchyme expressed both genes, Snail1 and Snail2 (FIGS. 2f and 2i), and, again, the epithelialisation thereof correlated with their repression and with the beginning of cadherin-16 expression (FIG. 2j-2o). On day 17.5 dpc, in addition to the facts mentioned regarding the collecting ducts, strong cadherin-16 expression was observed in the neuron (FIG. 2p-2u). At this time, the Snail genes are completely repressed in the kidney, without an undifferentiated mesenchyme being observed, a situation which remains throughout adult life. The data showed that the expression patterns are complementary in the different embryo stages and that Cadherin-16 only appeared from mesenchyme following the disappearance of the expression of the Snail1 and Snail2 genes. These data are consistent with the fact that Snail represses Cadherin-16 gene expression in vivo.

In order to determine whether Snail directly repressed the cadherin-16 gene transcription, its promoter was analysed; there, two Snail transcription factor-binding consensus E-boxes, located on-site in positions −581 and −746 (FIG. 2v), were found. When Snail1 or Snail2 were transfected jointly with a gene construct containing these two boxes and capable of reproducing the Cadherin-16 expression pattern (Whyte DA et al., 1999; Shao X et al., 2002), a 61% and 56% reduction was observed, respectively, in the promoter activity. In order to confirm whether the Snail binding boxes were necessary to produce this effect, constructs which had these boxes mutated or eliminated were co-transfected with Snail1 or Snail2. These experiments produced the same results, which indicates that Snail represses the Cadherin-16 promoter activity indirectly (FIG. 2v).

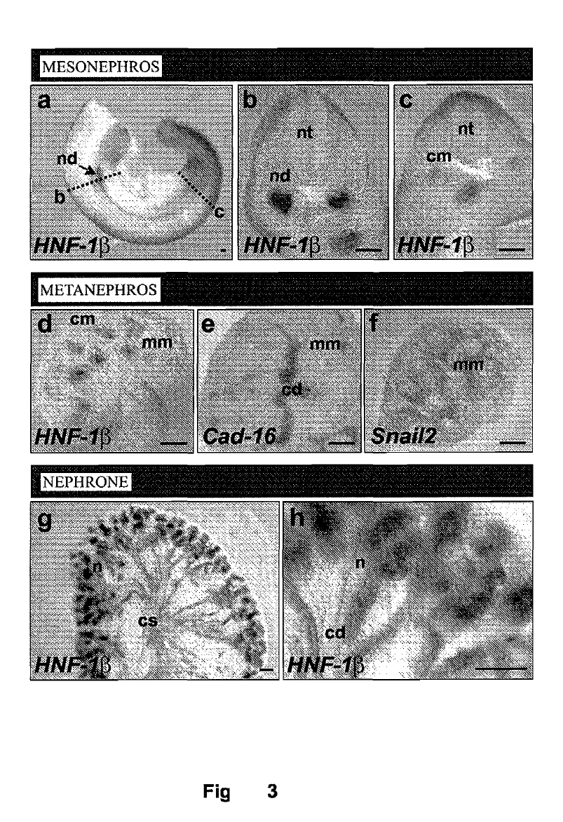

Since the Snail genes are characterised as repressors, we studied whether their repressor effect took place through the repression of a Cadherin-16 activator. HNF-1beta is a known potent Cadherin-16 activator (Bai et al., 2002) and, furthermore, it exhibits a very interesting expression pattern during kidney development. Thus, we studied its expression pattern in relation to that of the Snail genes and Cadherin-16, and an expression complementary to that of the Snail genes was observed, which appears-appeared when the Snail genes disappeared; it was also observed that the expression thereof occurs prior to that of Cadherin-16 and in the same regions. Thus, at 10 dpc, HNF-1beta is expressed in the differentiation of the nephric ducts in the posterior region (FIG. 3c), where the Snail genes have been repressed and cadherin-16 is not expressed (FIG. 2c). At 13.5 dpc, the metanephric mesenchyme expressed Snail1 and Snail2, whereas the HNF-1beta transcripts were detected in some areas of epithelialised mesenchyme which are still negative for cadherin-16 (FIG. 3d-f). The nephrons that differentiated from that mesenchyme to give rise to the functional epithelial structures of the mature kidney expressed both HNF-1beta and cadherin (FIG. 2). These data are consistent with the fact that Snail represses HNF-1 beta and that this repression prevents the appearance of cadherin-16.

In order to determine whether the acute activation of Snail is capable of repressing HNF-1beta and cadherin-16, an inducible construct that activates Snail1 by the administration of 4-OH-Tamoxifen was used (similar to that used in Locascio et al., 2002) in NMuMG cells. This method is used for the quick activation of transcription factors, since the protein is synthesised in an inactive form and the inducing agent simply transports it to the cell nucleus so that it may act as a transcription factor regulating the expression of other genes. These experiments made it possible to state that the activation of the Snail1 protein was sufficient to repress HNF-1 beta and cadherin-16 transcription in 24 h, thereby inducing a complete EMT (FIGS. 4a and 4c). Interestingly, 6 hours after Snail activation, a 20% repression of HNF-1beta levels was observed, together with unaltered cadherin-16 values. These data indicate that Snail induces the sequential repression of HNF-1 beta and cadherin-16, i.e., that Snail inhibits cadherin-16 expression through the repression of HNF-1 beta.

In order to determine whether HNF-1 beta repression by Snail takes place directly on the promoter, similar experiments to those conducted on the Cadherin-16 promoter were performed with Snail1 and Snail2. Two Snail-binding consensus E-boxes were identified, one of them conserved in the mouse and human promoter (FIG. 4d). The co-transfection experiments with the construct of this intact HNF-1 beta promoter showed that both genes, Snail1 and Snail 2—as previously described for the cadherin-16 promoter—, repressed the promoter activity (FIG. 4d), but this is not the case with those wherein the two boxes, or simply the conserved box, had been eliminated (FIG. 5d). These data show that Snail is a direct repressor of HNF-1beta gene transcription by binding with the conserved box.

### Example 2

### Snail Induces Renal Fibrosis in Transgenic Mice

In order to determine whether the Snail genes can repress HNF-1beta transcription and, consequently, the expression of Cadherin-16 and also of E-Cadherin (its known target in other tissues; reviewed in Barrallo-Gimeno and Nieto, 2005) in the kidney, transgenic mice with inducible Snail1 activity were generated (transgSnail1-ER mouse). The same system as for inducible expression in cell lines was used. Normal and transgenic mice were treated after birth with an injection of excipient or with tamoxifen for two weeks, after which they were sacrificed and the expression (FIG. 5c) and the location of the transgenic protein (FIGS. 5a and 5b) were analysed. The analysis of the corresponding kidneys showed the translocation of the exogenous protein to the nucleus (FIG. 5b) and the repression of E-Cadherin, Cadherin-16 and HNF-1beta gene expression only in the transgenic mice treated with tamoxifen (FIG. 5d-5i; supplementary FIG. 5 online). Also observed was the induction of the expression of Snail2, the member of the family that has prominent expression during kidney development (FIG. 5j-5l; supplementary FIG. 5 online) and has been shown to be functionally equivalent to Snail1 in cell lines and in embryos as an EMT inducer (Bolos et al., 2003; del Barrio and Nieto, 2002). In summary, it was observed that Snail1 represses HNF-1 beta in vivo, which induces the repression of cadherin-16. Jointly with this repression of E-cadherin, Snail1 expression has a great impact on the loss of epithelial characteristics, such that the marrow collecting duct cells seem to acquire a fibroblastic-like morphology reminiscent of EMT (FIGS. 5b, e, h, k, inserts).

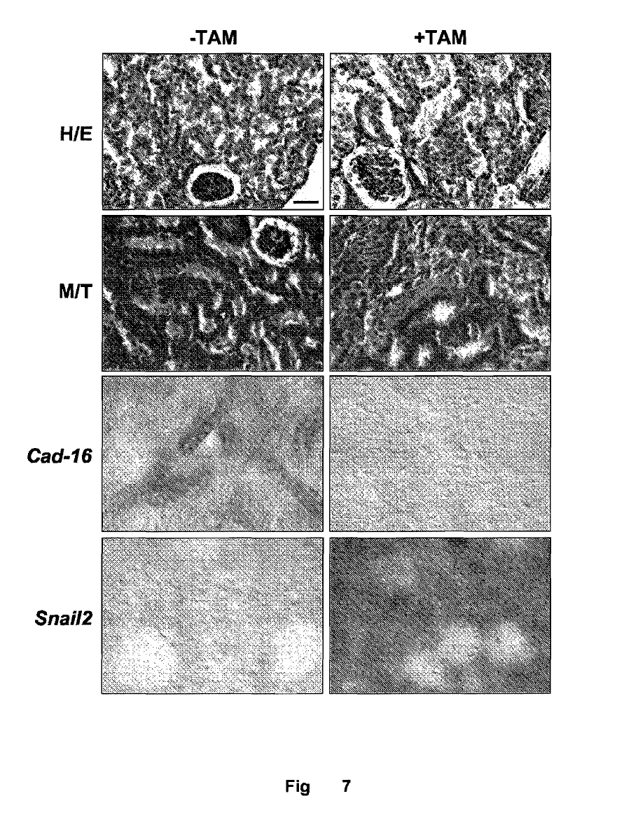

In order to verify that the expression of the Snail genes not only induced the repression of the above-mentioned genes, but a complete EMT, the histological phenotype of these transgenic animals' kidneys was analysed, and it was observed that the activation of Snail1 induced a morphological change in the kidney marrow consistent with EMT, activation of the mesenchymal marker vimentin and activation of Collagen I transcription (FIG. 6e-f), jointly with the deposits thereof (FIG. 6g-h), a prototypical marker of renal fibrosis (Alexakis et al., 2005). These changes also appeared in the renal cortex (FIG. 7). The changes described herein are reminiscent of those observed following the experimental induction of renal fibrosis under different conditions (Liu, 2003). These data indicate that the Snail1 and Snail2 genes act as repressors of the epithelial phenotype in the kidney and, moreover, that the activation thereof is sufficient to induce all the characteristics of renal fibrosis.

### Example 3

### The Snail Genes Pathologically Activate During the Human Renal Fibrosis Process

Samples of normal and fibrotic renal tissue from human kidneys obtained from patients subject to nephrectomy, more specifically, of renal tissue obtained from non-tumour renal areas in patients subject to nephrectomy (4 cases), and of fibrotic and non-fibrotic areas in patients suffering from renal failure caused by urinary obstruction, were analysed. The samples, of approximately 1 cm³, were fixated in 10% formalin or immediately frozen with isopentane in liquid nitrogen. The Research Ethics Committee of the Sant Joan Hospital of Alicante, where the samples were obtained, approved the protocols to analyse the tissues, which were subject to real-time RT-PCR analysis.

The quantitative RT-PCR was performed using the ABI PRISM® 7000 detection system and TaqMan® probes. The oligos and the probes were obtained from Applied Biosystems Assays-on-Demand, from the catalogue: Hs 99999905-m1 (GAPDH), Hs 00193591-m1 (SNAI1), Hs 00161904-m1 (SNAI2), Hs 00187880-m1 (CADH16). The RNA expression was calculated using the comparative Cₜ method normalised with the GAPDH levels. The data are expressed in relation to a calibrator (mock or wild mouse cells) using the formula 2^{-(ΔΔCt)}±SD. The RNAs were extracted and the cDNA was synthesised from the human kidney samples.

The histological characteristics of renal fibrosis were observed in the samples from patients suffering from renal failure (data not shown). On the contrary, no Snail1 or Snail2 transcript expression was observed in the normal human tissue (FIG. 7; n=4), whereas high Snail1 levels were detected in patients with renal fibrosis (FIG. 7).

As in the case of the animal models, Snail2 was expressed in the fibrotic tissue. Interestingly, the presence of fibrotic and non-fibrotic tissue in the same kidney shows that Snail activation is only associated with fibrosis (FIG. 7). In conclusion, it may be affirmed that human renal fibrosis is accompanied by the aberrant activation of Snail expression, leading to significant effects in the kidney's epithelial homeostasis. Snail1 activation in the adult kidney was sufficient to cause the epithelium-to-mesenchyme transition of the tubular cells and the collecting ducts, and to favour the deposit of collagen; therefore, Snail may be considered to be a good candidate for a therapeutic target or a prognostic and diagnostic marker of fibrosis and cyst formation that takes place in human renal diseases.

### Materials and Methods

Plasmids and antibodies. Expression plasmids. pcDNA3-Snail1 corresponds to the complete mouse Snail1 cDNA sequence inserted into plasmid pcDNA3 (Invitrogen; Cano et al., 2000). pcDNA3-Snail-ER corresponds to the Snail1-encoding sequence joined to a mutated version of the binding domain to the human estrogen receptor agonist that recognises the 4′-OH-Tamoxifen synthetic ligand (Locascio et al., 2002). The complete mouse Snail2 cDNA sequence was also cloned in pcDNA3 (pcDNA3-Snail2). Reporter plasmids. The pKsp(1268F)-Luc reporter construct of the mouse cadherin-16 promoter was generously supplied by Doctor Peter Igarashi (University of Texas, Southwestern Medical Center, Dallas, Tex.). The mouse HNF-1β promoter sequence, containing 1,072 pb from the ATG, was amplified by PCR from the genomic DNA of NMuMG cells using high-fidelity DNA polymerase (PfuTurbo, Stratagene). The primer oligonucleotides used for the amplification were 5′-ggtaccATCTACACATTCACTACTAGA-3′ (SEQ ID NO 10) and 5′-acgcgtTTTCCAAGGACGGAAAAAGAA-3′ (SEQ ID NO 11), corresponding to the GenBank™ X55842 sequence and containing the Kpnl and Mlul restriction sites at the 5′ ends, respectively. The purified PCR product was subcloned in vector pGL3-basic (Promega). The Quickchange Site Directed Mutagenesis kit (Stratagene) was used to introduce mutations inside the E-boxes present in the mouse cadherin-16 promoter. The 5′-CA(G/C)(G/C)TG-3′ sequence was mutated by 5′-AA(G/C)(G/C)TA-3′. The sequences of the oligonucleotides used to eliminate the E-boxes in the cadherin-16 and HNF-1β promoters are available upon request. Antibodies: Anti-E-cadherin (ECCD2, 1:200, Takara), anti-vimentin (M0725, 1:200, Dako). The Snail1-ER fusion protein was detected by immunoblots or immunohistochemistry using an anti-α human estrogen receptor antibody (1:100, Santa-Cruz). F-actin was detected using phalloidin-FITC (1:10, Sigma).

Cell culture and generation of stably transfected cells with Snail1 or Snail1-ER. The NMuMG cell line comes from the epithelium of mouse mammary gland. This epithelial cell line expresses high E-cadherin levels and is very sensitive to the epithelium-mesenchyme transition induced by TGFβ (Miettinen et al., 1994). For the transfection, the NMuMG cells were seeded in 6-well plates (5×10⁴ cells per 3.5-cm well) in a 1:1 mixture of Ham's F12 medium and Dulbecco's Modified Eagle's medium supplemented with 100 IU/ml of penicillin, 0.1 mg/ml of streptomycin, 2 mM of glutamine and 2.5 μg/ml of amphothericin B, 10% of fetal bovine serum and 10 μg/ml of insulin. 24 hours after seeding the cells, 500 ng of DNA were added in the presence of LIPOFECTAMINE (Roche) and the cells were incubated with the DNA overnight. One day later, selection of the neomycin-resistant transfected cells began, using neomycin analogue G-418 (Calbiochem, 400 μg/ml). Two independent clones transfected with Snail1 or Snail-ER were isolated.

Migration and invasion assays. The cells were seeded in 6-well plates at a density of 3×10⁵ cells per well. After 24 hours, a wound was made in the central area of the confluent culture and the cells were incubated for an additional 24-hour period after washing the culture and adding fresh medium thereto. The cultures were observed at several times and photographs were taken of the wounded area using a Zeiss Axiovert inverted microscope. The invasion assays in type IV collagen gels were performed in Boyden chambers, as described in Cano et al., 2000. The cells in the lower compartment were collected and counted after 24 hours. Parallel to this, the nuclei of the cells on the lower part of the filter were stained with DAPI following fixation in methanol and after having removed all the cells from the upper part of the filter.

Microarray genetic analysis. The Affymetrix murine genome Chip U74Av2 was used to define the gene expression profile of the cells transfected with Snail1 (Snail1-transfectants). This profile was compared to that of the cells transfected with the empty vector (mock-transfectants). The biotinylated RNAs were analysed and hybridized to the Chip. Subsequently, the Chip was stained with streptavidin-phycoerithrin and scanned. The chips were analysed using the Affymetrix® Microarray Suite 5.0 programme.

Transcript analysis. The Poly(A)+ mRNAs were extracted from the NMuMG cells using the Microfast Track kit (Invitrogen). For the Northern blot analysis, 1.5-μg aliquots of Poly(A)+ mRNAs purified with oligo(dT) cellulose were transferred to nylon membranes that were hybridized with [α-³²P]dCTP-radiolabelled probes (rediprime II, Amersham Biosciences). The Snail1, E-cadherin, cadherin-16 and GAPDH DNA probes were amplified by RT-PCR from 25 ng of the corresponding purified cDNAs and the hybridizations thereof were visualised by autoradiography using a Hyperfilm MP (Amershan Biosciences). The RT-PCR for Snail1 and GAPDH was described in Cano et al., 2000. The real-time RT-PCR was performed using the ABI PRISM® 7000 sequence detection system and TaqMan® probes. The oligonucleotides and the probes were obtained from Applied Biosystems Assays-on-Demand, as follows: Mm-99999915-g1 (GAPDH), Mm-00441533-g1 (Snail1), Mm00483196-m1 (Cadherin-16) and Mm-00447452-m1 (HNF-18). The RNA expression was calculated using the comparative Cₜ method normalised with GAPDH. The final results are expressed in relation to a calibrator (mock or wild mouse cells) using the formula 2^{-(ΔΔCt)}±SD. The RNA was extracted and the cDNA was synthesised from the cells transfected with Snail1-ER or with the empty vector at several times following treatment with 4′-OH-Tamoxifen and from the kidneys of the newborn normal or transgenic animals or two weeks after the administration of tamoxifen or of an excipient.

In situ hybridization. The mouse embryos came from the Balb-C strain, and their ages, established in days post-coitum (dpc) were determined considering the day when the vaginal plug is seen as day 0.5. The urogenital systems or the kidneys were desiccated at 13.5 dpc and 17.5 dpc, respectively, and fixated in 4% paraformaldehyde in PBS/DEPC overnight. Subsequently, they were processed directly for in situ hybridization (ISH) or soaked in gelatin and cut with a vibratome in order to obtain 50-μm sections. The ISHs in gelatin sections or in intact embryos were performed as described in Blanco et al., 2002 using the DIG-11-UTP-labelled mouse Snail1, Snail2 and E-cadherin RNA probes (Cano et al., 2000; Sefton et al., 1998). The probes for mouse Cadherin-16 and HNF-1β were obtained by RT-PCR from the cDNA of the NMuMG cells using the oligonucleotides described above. Following the hybridization, the embryos or the kidney sections were processed as described in Cano et al., 2000.

Promoter analysis. The activity of the cadherin-16 and HNF-1β promoters was determined by co-transfecting the NMuMG cells with 50 ng of pcDNA3-Snail, pcDNA3-mSnail2 or the empty vector, and with 300 ng of pKsp(1268F)-Luc or 400 ng of pmHNF-1β-Luc. A plasmid with the Renilla reniformis luciferase gene (phRL-CMV-Luc, Promega) was co-transfected as an efficiency control. 24 hours after the transfection, the activity of the firefly (Luc) and Renilla luciferases was determined using the Dual Luciferase Reporter Assay system (Promega), following the supplier's instructions. The Luc activity was normalised to that of the Renilla luciferase. In all the experiments, the total quantity of transfected DNA was standardised by adding the empty vector. The results are represented as the percentage of luciferase activity relative to the controls (luciferase values in cells co-transfected with the empty vector).

Transgenic mice. The Snail1-ER transgene was designed as previously described (Locascio et al., 2002), and a transgenic mouse (transgSnail1-ER mouse) was generated for this construct following standard procedures (Hogan, B., Beddington, R. and Lacy, F. Manipulating the mouse embryo. A laboratory manual. Cold Spring Harbor Laboratory Press (1994)). For this study, we selected an animal line that had a very high expression of the transgenic protein in the kidney. In this model, even though the Snail1-ER protein is constitutively expressed, its function as a transcription factor develops only when the protein is translocated in the nucleus following treatment with tamoxifen. The transgene is detected from the animal tails' DNA by PCR (the details about the oligonucleotides used are available upon request). The protein's subcellular location was analysed by immunohistochemistry using an anti-human estrogen receptor antibody. The same antibody was used to assess the quantity of Snail1-ER protein in the different tissues from the transgenic mice by Western Blots. The tamoxifen (Sigma) was first dissolved in ethanol (10% of the final volume) and, subsequently, in corn oil (Sigma) in order to obtain a final concentration of 30 mg/ml. The solution was sonicated in order to improve its solubility and 3 mg of Tamoxifen for every 20 grams of body weight were administered subcutaneously to the newborn animals every three days for two weeks. Once the treatment was concluded, the animals were sacrificed and the kidneys obtained were soaked in gelatin, cut with a vibratome for the ISH or immunohistochemistry or processed for the extraction of RNAs.

### REFERENCES

Bai, Y., Pontoglio, M., Hiesberger, T., Sinclair, A. M. & Igarashi, P. Regulation of kidney-specific Kspcadherin gene promoter by hepatocyte nuclear factor-1beta. Am. J. Physiol. Renal Physiol. 283, F839-51 (2002).
Barrallo-Gimeno, A. and Nieto, M. A. The Snail genes as inducers of cell movement and survival: implications in development and cancer. Development 132, 3151-61 (2005).
Bathe, E. et al. The transcription factor snail is a repressor of E-cadherin gene expression in epithelial tumour cells. Nat. Cell Biol. 2, 84-9 (2000).
Bolos, V. et al. The transcription factor Slug represses E-cadherin expression and induces epithelial to mesenchymal transitions: a comparison with Snail and E47 repressors. J. Cell Sci. 116, 499-511 (2003).
Cano, A. et al. The transcription factor snail controls mesenchymal-epithelial transitions by repressing Ecadherin expression. Nat. Cell Biol. 2, 76-83 (2000).
Chilosi et al., 2003.
del Barrio, M. G. and Nieto, M. A. Overexpression of Snail family members highlights their ability to promote chick neural crest formation. Development 129, 1583-93 (2002).
Dressler, 2002. Book.
Hawker, C. J. and Wooley, K. L. The convergence of synthetic organic and polymer chemistries. Science 19 (309): 1200-5 (2005).
Huber et al. Current Op Cell Biol (2005).
Iwano, M. et al. Evidence that fibroblasts derive from epithelium during tissue fibrosis. J. Clin. Invest. 110, 341-50 (2002).
Jinde et al.
Kalluri and Neilson, 2003.
Li et al., 2005.
Locascio, A., Vega, S., de Frutos, C. A., Manzanares, M. and Nieto, M. A. Biological potential of a functional human SNAIL retrogene. J. Biol. Chem. 277, 38803-9 (2002).
Rastaldi et al., 2002.
Lu, P. V. and Woodle, M. C. In vivo application of RNA interference: from functional genomics to therapeutics. Adv Genet 54: 117-42 (2005).
Sato, M., Muragaki, Y., Saika, S., Roberts, A. B. and Ooshima, A. Targeted disruption of TGF-beta1/Smad3 signaling protects against renal tubulointerstitial fibrosis induced by unilateral ureteral obstruction. J. Clin. Invest. 112, 1486-94 (2003).
Shao, X., Johnson, J. E., Richardson, J. A., Hiesberger, T. and Igarashi, P. A minimal Ksp-cadherin promoter linked to a green fluorescent protein reporter gene exhibits tissue-specific expression in the developing kidney and genitourinary tract. J. Am. Soc. Nephrol. 13, 1824-36 (2002).
Thompson, R. B. et al. Isolation and cDNA cloning of Kspcadherin, a novel kidney-specific member of the cadherin multigene family. J. Biol. Chem. 270, 17594-601 (1995).
Vongwiwatana et al., 2005.
Whyte, D. A. et al. Ksp-cadherin gene promoter. I. Characterization and renal epithelial cell-specific activity. Am. J. Physiol. 277, F587-98 (1999).
Yáñez-Mo et al., 2002.
Zeisberg, M. and Kalluri, R. The role of epithelial-to-mesenchymal transition in renal fibrosis. J. Mol. Med. 82, 175-181 (2004).

1. A method of identifying renal fibrosis in a patient comprising the following steps:
a) identifying the presence of Snail, in a biological sample of renal tissue from the patient, and
b) comparing the presence of Snail observed in step a) with the absence thereof in a normal human kidney tissue; wherein the presence of Snail is indicative of the existence of renal fibrosis.

2. The method according to claim 1, wherein the Snail of step a) is the human Snail 1 gene (SEQ ID NO 5) or the Snail2 gene (SEQ ID NO 7) transcript.

3. The method according to claim 1, wherein the Snail of step a) is the human Snail 1 protein (SEQ ID NO 6) or the human Snail2 protein (SEQ ID NO 8).

4. The method according to claim 1, wherein step a) comprises identifying the presence of Snail using specific monoclonal or polyclonal Snail antibodies.

5. The method according to claim 1, wherein step a) comprises identifying the presence of Snail using in situ hybridization with a Snail precursor.

6. The method according to claim 1, wherein step a) comprises identifying the presence of Snail using RT-PCR amplification of a Snail gene precursor.

7. A method of identifying and evaluating the activity of Snail protein inhibitory compounds useful for the treatment of renal fibrosis comprising the following steps:
a) incubating, under suitable conditions, a compound with a biological system expressing Snail, wherein the presence of Snail in the biological system produces renal fibrosis;
b) measuring an indicative parameter of renal fibrosis; and
c) identifying a compound that inhibits Snail protein activity wherein the activity comprises a reduction of the renal fibrosis parameter.

8. The identification method according to claim 7, wherein the biological system of step a) is a transgenic animal, wherein the expression of the Snail protein is inducible in a constant or conditional manner and wherein the expression of the Snail protein causes renal fibrosis.

9. The identification method according to claim 8, wherein the transgenic animal is a transgSnail1-ER mouse.

10. The identification method according to claim 7, wherein the parameter related to renal fibrosis of step b) belongs to the group consisting of a morphological change characteristic of an EMT, level of vimentin, Collagen I gene transcription, and deposition of collagen fibres.

11. A method for treating renal fibrosis comprising administering to a patient in need thereof a therapeutically effect amount of a compound or agent that inhibits Snail protein activity.

12. The method according to claim 11, wherein the compound or agent is a nucleic acid or polynucleotide which prevents or reduces the expression of the human Snail protein encoding gene and includes a nucleotide sequence selected from the group consisting of:
a) a Snail protein gene or mRNA sequence specific anti-sense nucleotide sequence,
b) a Snail protein mRNA specific ribozyme,
c) a Snail protein mRNA specific aptamer, and
d) a Snail protein mRNA specific interference RNA (iRNA).

13. The method according to claim 12, wherein the iRNA is bound to the Snail mRNA fragment sequence (SEQ ID NO 9) or to another fragment that comprises SEQ ID NO 9.

14. A pharmaceutical composition for the treatment of renal fibrosis comprising a therapeutically effective quantity of a compound or agent that inhibits Snail protein.

15. The pharmaceutical composition according to claim 14, wherein the inhibitory compound is a nucleic acid or polynucleotide and which prevents or reduces the expression of the human Snail protein encoding gene.

16. The pharmaceutical composition according to claim 20, wherein the iRNA is bound to the Snail mRNA fragment sequence (SEQ ID NO 9) or to another fragment that comprises SEQ ID NO 9.

17. A method for treating renal fibrosis comprising administering to a patient in need thereof a therapeutically effect amount of the pharmaceutical composition according to claim 14.

18. The method according to claim 17, wherein the renal fibrosis is caused by a disease, disorder or pathology selected from the consisting of glomerulonephritis, IgA nephropathy, diabetes, renal damage induced by toxicity, urinary obstruction, and deterioration of a kidney transplant.

19. The pharmaceutical composition of claim 14, further comprising one or more pharmaceutically acceptable adjuvants or vehicles.

20. The nucleotide sequence of claim 15, wherein the nucleotide sequence belongs to the group consisting of:
a) a Snail protein gene or mRNA sequence specific anti-sense nucleotide sequence,
b) a Snail protein mRNA specific ribozyme,
c) a Snail protein mRNA specific aptamer, and
d) a Snail protein mRNA specific interference RNA (iRNA).
